(19) United States
(12) Patent Application Publication
(10) Pub. No.: US 2013/0131126 A1
(43) Pub. Date: May 23, 2013

(54) DERIVATIVES OF 1,4-DIHYDROPYRIDINE POSSESSING ANTIVIRAL EFFICACY

(71) Applicants: Ilmars Stonans, Riga (LV); Ilze Jansone, Riga (LV); Indra Jonane-Osa, Riga (LV); Egils Bisenieks, Riga (LV); Gunars Duburs, Riga (LV); Ivars Kalvins, Ikskile (LV); Brigita Vigante, Riga (LV); Janis Uldrikis, Jelgava (LV); Imanta Bruvere, Riga (LV); Liga Zuka, Riga (LV); Janis Poikans, Riga (LV); Zaiga Neidere, Riga (LV)

(72) Inventors: Ilmars Stonans, Riga (LV); Ilze Jansone, Riga (LV); Indra Jonane-Osa, Riga (LV); Egils Bisenieks, Riga (LV); Gunars Duburs, Riga (LV); Ivars Kalvins, Ikskile (LV); Brigita Vigante, Riga (LV); Janis Uldrikis, Jelgava (LV); Imanta Bruvere, Riga (LV); Liga Zuka, Riga (LV); Janis Poikans, Riga (LV); Zaiga Neidere, Riga (LV)

(73) Assignee: JSC Grindeks, Riga (LV)

(21) Appl. No.: 13/810,345

(22) PCT Filed: Jul. 15, 2011
(86) PCT No.: PCT/EP11/03526

§ 371 Date: Feb. 5, 2013

(30) Foreign Application Priority Data
Jul. 16, 2010 (EP) 10169758.9
Jul. 16, 2010 (EP) 10169759.7
Jul. 16, 2010 (EP) 10169760.5

(51) Int. Cl. C07D 211/90

(52) U.S. Cl. CPC C07D 211/90
USPC 514/356; 546/321

(57) ABSTRACT

2,6-dimethyl-1,4-dihydropyridine-3,5-dicarboxylic acid ester type compounds having general formula I

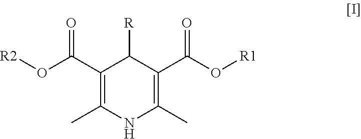

wherein
R is hydrogen or carboxylate-methyl ester,
R₁ is sodium carboxylate-methyl ester
R₂ is methyl, ethyl or sodium carboxyate-methyl ester possessing antiviral efficacy.

### TECHNICAL FIELD

The present invention relates to new 2,6-dimethyl-1,4-dihydropyridine-3,5-dicarboxylic acid ester type compounds having general formula I

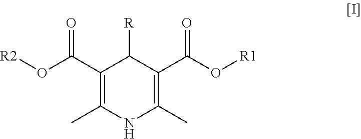

wherein
R is hydrogen or carboxylate-methyl ester
R₁ is sodium carboxylate-methyl ester
R₂ is methyl, ethyl or sodium carboxylate-methyl ester

New 2,6-dimethyl-1,4-dihydropyridine-3,5-dicarboxylic acid ester type compounds with general formula I possessing antiviral activity.

### BACKGROUND ART

Influenza, commonly called “the flu,” is an illness caused by RNA viruses that infect the respiratory tract of many animals, birds, and humans. In most people, the infection results in the person getting fever, cough, headache, and malaise (tired, no energy); some people also may develop a sore throat, nausea, vomiting, and diarrhea. The majority of individuals has symptoms for about one to two weeks and then recovers with no problems. However, compared with most other viral respiratory infections, such as the common cold, influenza (flu) infection can cause a more severe illness with a mortality rate (death rate) of about 0.1% of people who are infected with the virus. Some influenza viruses develop resistance to the antiviral medicines, limiting the effectiveness of treatment.

There are known some medicaments which are effective against influenza viruses, such as 1-methyl-2-phenylthiomethyl-3-carbethoxy-4-dimethylaminomethyl-5-oxybromoindole monohydrate hydrochloride (is marketed under the trade name Arbidol®), Oseltamivir (is marketed by Roche under the trade name Tamiflu®) and others.

Oseltamivir is indicated for the treatment and prevention of infections due to influenza A and B virus. Oseltamivir was disclosed in EP 0759917 B (GILEAD SCIENCES INC) Dec. 4, 2000.

GB 2234510 A (NAUCHNO-ISSLEDOVATELSKY INSTITUT MEDITSINSKOI RADIOLOGII AKADEMII MEDITSINSKIKH NAUK SSSR) Jun. 2, 1991 disclosed 1-methyl-2-phenylthiomethyl-3-carbethoxy-4-dimethylaminomethyl-5-oxybromoindole monohydrate hydrochloride as an active agent in a pharmaceutical preparation of antiviral, interferon inducing and immunomodulating action.

The most similar to given compound with general formula I is disodium salt of 2,6-dimethyl-1,4-dihydropyridine-3,6-bis-carbonyloxyacetic acid with anti-arrhythmic action, which was disclosed in DUBUR, et al. Anti-arrhythmic action of preparations of the dihydropyridine series. Farmakol. Toksikol. 1983, vol. 46, no. 6, p. 20-24.

### DISCLOSURE OF INVENTION

An object of the present invention is to provide new compounds, possessing antiviral activity and process for preparing them.

The above-mentioned object is attained by providing new 2,6-dimethyl-1,4-dihydropyridine-3,5-dicarboxylic acid ester type compounds having general formula I

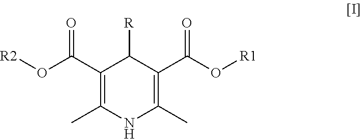

wherein
R is hydrogen or carboxylate-methyl ester
R₁ is sodium carboxylate-methyl ester
R₂ is methyl, ethyl or sodium carboxylate-methyl ester

The compounds according of general formula I are:
sodium 2,6-dimethyl-3-ethoxycarbonyl-1,4-dihydropyridine-5-carbonyloxyacetate (formula VI):

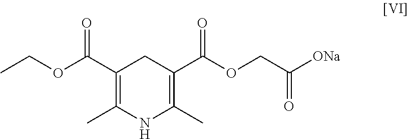

sodium 2,6-dimethyl-3-methoxycarbonyl-1,4-dihydropyridine-5-carbonyloxyacetate (formula X):

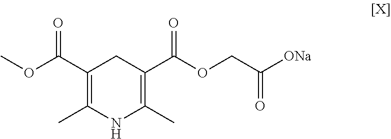

disodium 2,6-dimethyl-1,4-dihydropyridine-4-methoxycarbonyl-3,5-bis-(carbonyloxyacetate) (formula XV):

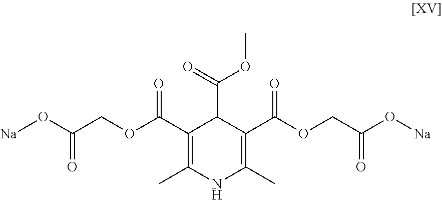

The compounds possess antiviral activity, due this properties new 2,6-dimethyl-1,4-dihydropyridine-3,5-dicarboxylic acid ester type compounds having general formula I may be used in medicine. The new 2,6-dimethyl-1,4-dihydropyridine-3,5-dicarboxylic acid ester type compounds having general formula I can be use as a solution of injection and as tablets or other solid dosage forms.

An object of the present invention is a method of preparation of said compound of general formula I.

The common process for the preparation of 2,6-dimethyl-1,4-dihydropyridine-3,5-dicarboxylic acid ester type compounds comprises:
a) reacting of derivative of formaldehyde with derivative of acetoacetic acid ester and enamine in appropriate solvent, which is selected from the group of methanol, ethanol, propanol or butanol;
b) treatment with sodium hydroxide in appropriate solvent, which is selected from the group of methanol, ethanol, propanol or butanol.

The general process for the preparation of 2,6-dimethyl-1,4-dihydropyridine-3,5-dicarboxylic acid ester type compounds is represented bellow:

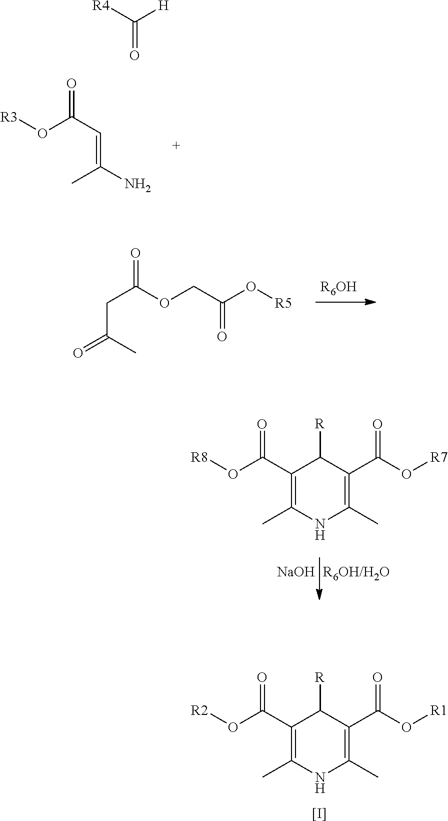

wherein
R is hydrogen or methoxycarbonyl
R₁ is sodium carboxylate-methyl ester
R₂ is methyl, ethyl or sodium carboxylate-methyl ester
R₃ is methyl, ethyl or diethoxycarbonylmethyl ester
R₄ is hydrogen or carboxyl
R₅ is methyl or ethyl
R₆ is methyl, ethyl, propyl or butyl
R₇ is carboxylate-methyl ester or diethoxycarbonylmethyl ester
R₈ is methyl, ethyl or diethoxycarbonylmethyl ester

### BRIEF DESCRIPTION OF DRAWINGS

FIG. 1. represented antiviral efficacy of Oseltamivir on MDCK (Madin-Darby Canine Kidney epithelial) cell line in vitro;

FIG. 2. represented antiviral efficacy of 1-methyl-2-phenylthiomethyl-3-carbethoxy-4-dimethylaminomethyl-5-oxybromoindole monohydrate hydrochloride on MDCK cell line in vitro;

FIG. 3. represented antiviral efficacy of sodium 2,6-dimethyl-3-ethoxycarbonyl-1,4-dihydropyridine-5-carbonyloxyacetate on MDCK cell line in vitro.

FIG. 4. represented antiviral efficacy of sodium 2,6-dimethyl-3-methoxycarbonyl-1,4-dihydropyridine-5-carbonyloxyacetate on MDCK cell line in vitro.

FIG. 5. represented antiviral efficacy of disodium 2,6-dimethyl-1,4-dihydropyridine-4-methoxycarbonyl-3,5-bis-(carbonyloxyacetate) on MDCK cell line in vitro.

The present invention will be described in more detail by referring to the following non-limiting examples.

### BEST MODE FOR CARRYING OUT THE INVENTION

### Example 1

### Preparation of sodium 2,6-dimethyl-3-ethoxycarbonyl-1,4-dihydropyridine-5-carbonyloxyacetate

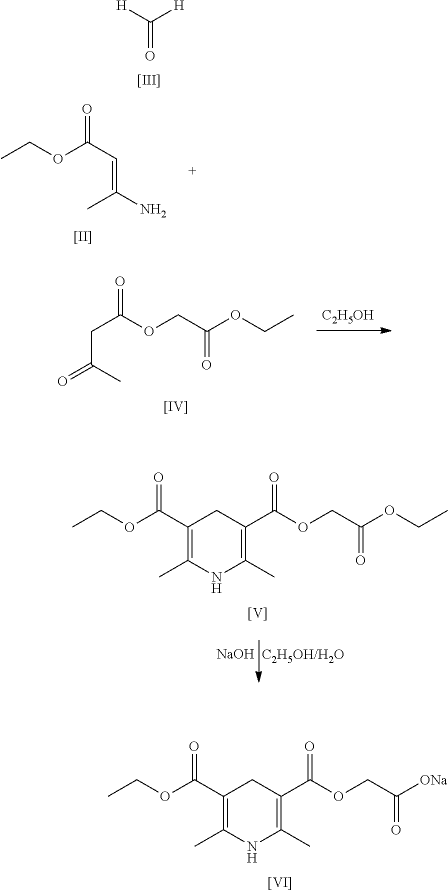

2-Ethoxycarbonyl methyl ester of acetoacetate (formula IV) (3.8 g, 20 mmol), ethyl-β-aminocrotonate (formula II) (2.7 g, 20 mmol) were dissolved in ethanol (10 mL). Formaldehyde (formula III) (2.6 mL, 20 mmol) was added to reaction mixture, which was heated thereafter for 1 hour and then cooled. The precipitates were separated by filtration. 2,6-Dimethyl-3-ethoxycarbonyl-5-(ethoxycarbonyl)methoxycarbonyl-1,4-dihydropyridine (formula V) was obtained as pale yellow crystalline powder.

2,6-Dimethyl-3-ethoxycarbonyl-5-(ethoxycarbonyl)methoxycarbonyl-1,4-dihydropyridine (formula V) was dissolved in ethanol (10 mL). Sodium hydroxide (0.2 g, 5 mmol), which was previously dissolved in water (5 mL), was added dropwise to reaction mixture. Reaction mixture was stirred for additional 10 minutes and thereafter cooled. The precipitates were separated by filtration.

Sodium 2,6-dimethyl-3-ethoxycarbonyl-1,4-dihydropyridine-5-carbonyloxyacetate (formula VI) was dried at ambient temperature. The yield of the sodium 2,6-dimethyl-3-ethoxycarbonyl-1,4-dihydropyridine-5-carbonyloxyacetate (formula VI) was 1.37 g (80%), having a melting temperature of 240-242° C.

¹H-NMR spectrum (400 MHz, DMSO-d₆, TMS) δ: 1.18 (3H, t, J=7 Hz, 3-COOCH₂CH₃); 2.11 (6H, s, 2.6-CH₃); 3.13 (2H, s, 4-H₂); 4.04 (2H, q, J=7 Hz, 3-COOCH₂CH₃); 4.15 (2H, s, 5-COOCH₂); 8.36 (1H, br.s., NH).

Elemental Analysis for C₁₃H₁₆NNaO₆×2H₂O: Found, %: C 45.85; H 5.76; N 4.00; Calculated, %: C 45.75; H 5.91; N 4.10.

### Example 2

### Preparation of sodium 2,6-dimethyl-3-methoxycarbonyl-1,4-dihydropyridine-5-carbonyloxyacetate

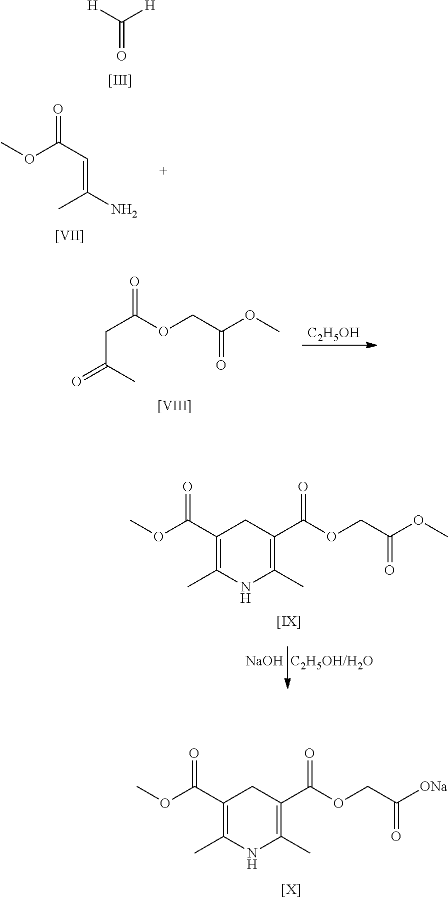

2-Methoxycarbonyl methyl ester of acetoacetate (formula VIII) (8.71 g, 50 mmol), methyl-β-aminocrotonate (formula VII) (5.75 g, 50 mmol) were dissolved in ethanol (25 mL). Formaldehyde (formula III) (6.5 mL, 50 mmol) was added to reaction mixture, which was heated thereafter for 1 hour and then cooled. The precipitates were separated by filtration. 2,6-Dimethyl-3-methoxycarbonyl-5-(methoxycarbonyl)methoxycarbonyl-1,4-dihydropyridine (formula IX) was obtained as a crystalline powder.

2,6-Dimethyl-3-methoxycarbonyl-5-(methoxycarbonyl)methoxycarbonyl-1,4-dihydropyridine (formula IX) was dissolved in ethanol (10 mL). Sodium hydroxide (0.68 g, 17 mmol), which was previously dissolved in water (5 mL), was added dropwise to reaction mixture. Reaction mixture was stirred for additional 10 minutes and thereafter cooled. The precipitates were separated by filtration. p Sodium 2,6-dimethyl-3-methoxycarbonyl-1,4-dihydropyridine-5-carbonyloxyacetate (formula X) was dried at ambient temperature. The yield of the sodium 2,6-dimethyl-3-methoxycarbonyl-1,4-dihydropyridine-5-carbonyloxyacetate (formula X) was 4.48 g (85%), having a melting temperature of 290-292° C.

¹H-NMR spectrum (400 MHz, DMSO-d₆, TMS) δ: 2.11 (6H, s, 2.6-CH₃); 3.14 (2H, s, 4-H₂); 3.57 (3H, s, 3-COOCH₂CH₃); 4.15 (2H, s, 5-COOCH₂); 8.41 (1H, br.s., NH).

Elemental Analysis for C₁₂H₁₄NNaO₆×H₂O: Found, %: C 46.35; H 4.84; N 4.35; Calculated, %: C 46.61; H 5.22; N 4.53.

Preparation of disodium 2,6-dimethyl-1,4-dihydropyridine-4-methoxycarbonyl-3,5-bis-(carbonyloxyacetate)

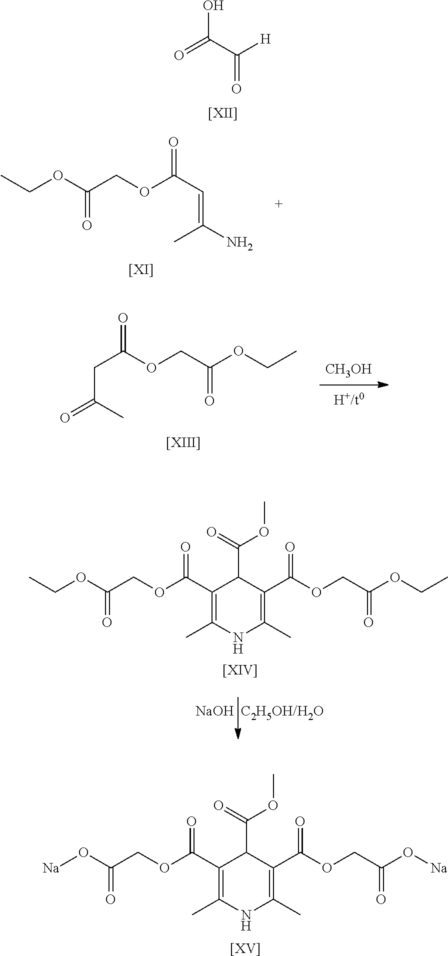

2-Ethoxycarbonyl methyl ester of acetoacetate (formula XIII) (20 mmol), 2-ethoxycarbonyl methyl ester of β-aminocrotonate (formula XI) (20 mmol) were dissolved in methanol (10 mL). Glyoxylic acid (formula XII) (20 mmol) was added to reaction mixture, which was heated thereafter for 1 hour and then cooled. The precipitates were separated by filtration. 2,6-Dimethyl-4-carbonicacid-3,5-(ethoxycarbonyl)methoxycarbonyl-1,4-dihydropyridine was obtained as pale yellow crystalline powder which (10 mmol) was dissolved into methanol (60 mL). Concentrated sulphuric acid (0.2 mL) was added to the reaction mixture. The reaction mixture was stirred and heated for 3 hours. The solvent was removed by distillation at a reduced pressure. The precipitates were separated by filtration. The obtained intermediate 2,6-dimethyl-1,4-dihydropyridin-4-methoxycarbonyl-3,5-tricarbonicacid 2,5-diethoxycarbonylmethyl ester (formula XIV) was dried at under reduced pressure. The yield was 3.7 g (95%).

2,6-Dimethyl-1,4-dihydropyridin-4-methoxycarbonyl-3,5-tricarbonicacid 2,5-diethoxycarbonylmethyl ester (formula XIV) was dissolved in ethanol (35 mL) and heated till 60° C. at which point aqueous sodium hydroxide (0.65 g, 16.1 mmol) was added dropwise. Reaction mixture was heated for 5 minutes and then cooled. The precipitates were separated by filtration. After the recrystallization of crude disodium 2,6-dimethyl-1,4-dihydropyridine-4-methoxycarbonyl-3,5-bis-(carbonyloxyacetate) (formula XV) from mixture of methanol and acetone, having a melting temperature of 260-262° C.

¹H-NMR spectrum (400 MHz, DMSO-d₆, TMS) δ: 2.20 (6H, s, 2,6-CH₃); 3.42 (3H, s, 4-OCH₃); 3.98 and 4.28 (4H, dd, —OCH₂O—); 4.76 (1H, s, 4-CH); 8.90 (1H, s, NH).

Elemental Analysis for C₁₅H₁₅NNa₂O₁₀×2.6H₂O: Found, %: C 39.61; H 4.23; N 2.85; Calculated, %: C 38.99; H 4.40; N 3.03.

Efficiency of 2,6-dimethyl-1,4-dihydropyridine-3,5-dicarboxylic acid ester type compounds having general formula I—sodium 2,6-dimethyl-3-ethoxycarbonyl-1,4-dihydropyridine-5-carbonyloxyacetate (formula VI), sodium 2,6-dimethyl-3-methoxycarbonyl-1,4-dihydropyridine-5-carbonyloxyacetate (formula X) and disodium 2,6-dimethyl-1,4-dihydropyridine-4-methoxycarbonyl-3,5-bis-(carbonyloxyacetate) (formula XV), against influenza viruses was evaluated on MDCK-Madin-Darby Canine Kidney epithelialcell line, in vitro.

For influenza virus, MDCK cells that were permissive of viral replication were grown up to sufficient numbers in growth media with supplements. Once MDCK cells were confluent they were seeded into 96 well flat-bottomed plates (2×10⁴ cells/well), incubated overnight and then infected with the influenza virus (H3N2) at the correct concentration and incubated in order to allow productive infection of the MDCK cells.

After the initial seeding of cells overnight (200 μl/well), the medium was removed and influenza viral infection performed in a smaller volume (25 μl/well) for 1 hour. After the 1 hour infection, the viral inoculum was removed and replaced with medium (200 μl/well) containing test compound. Thus, 2,6-dimethyl-1,4-dihydropyridine-3,5-dicarboxylic acid ester type compounds having general formula I—sodium 2,6-dimethyl-3-ethoxycarbonyl-1,4-dihydropyridine-5-carbonyloxyacetate, sodium 2,6-dimethyl-3-methoxycarbonyl-1,4-dihydropyridine-5-carbonyloxyacetate and disodium 2,6-dimethyl-1,4-dihydropyridine-4-methoxycarbonyl-3,5-bis-(carbonyloxyacetate), were presented from 1 hour after viral infection until the end of the culture period.

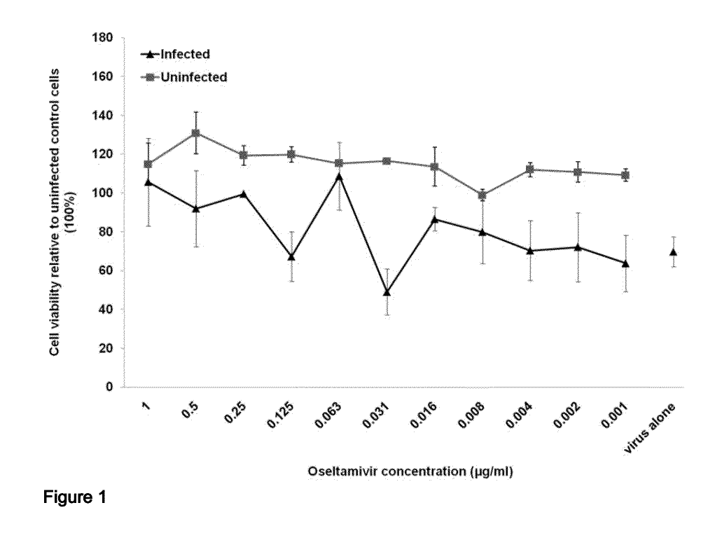

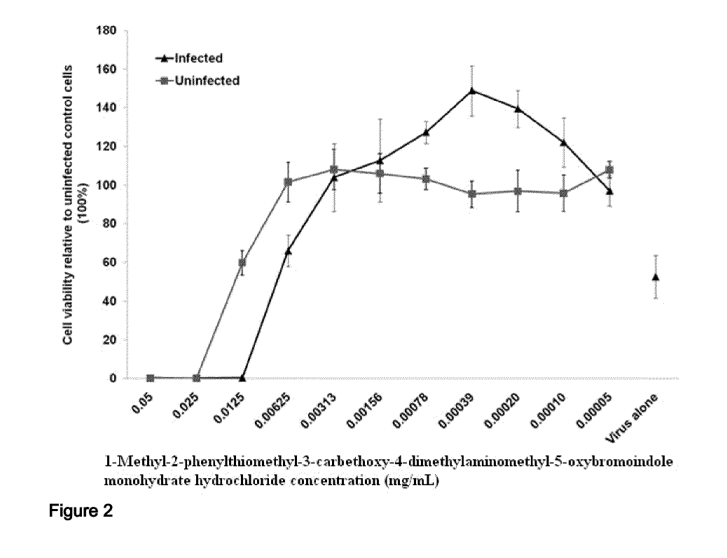

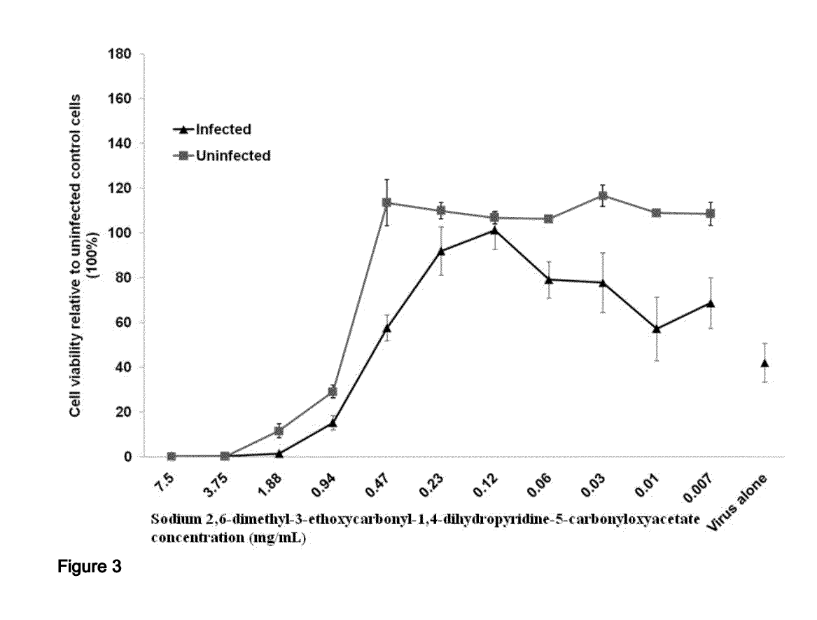

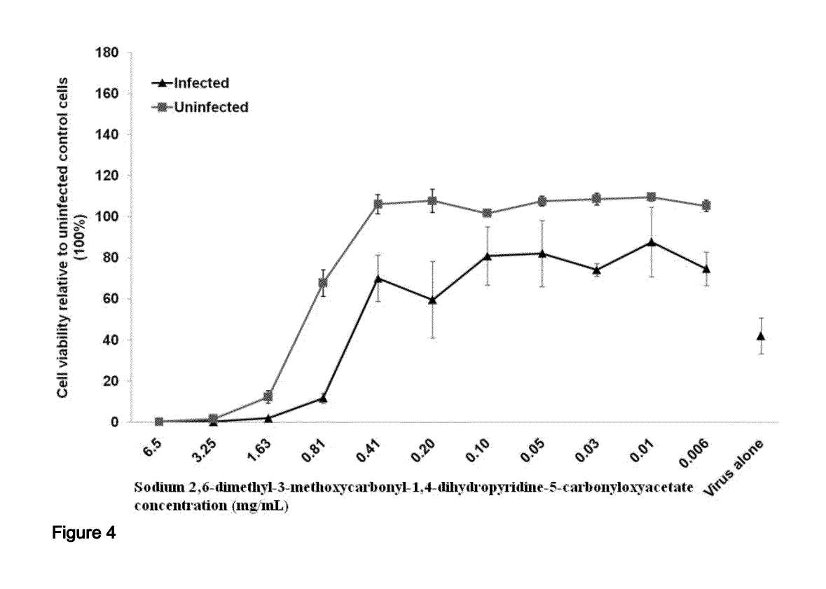

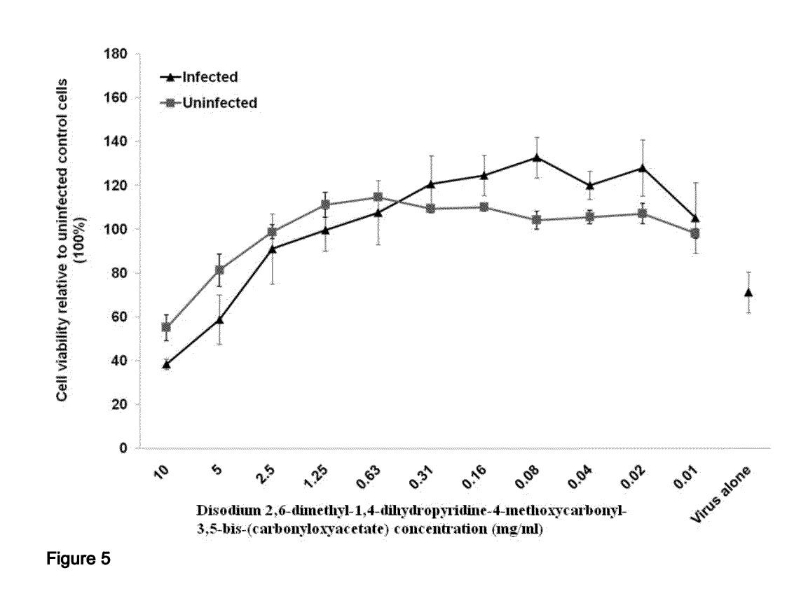

The antiviral efficacy of 2,6-dimethyl-1,4-dihydropyridine-3,5-dicarboxylic acid ester type compounds having general formula I—sodium 2,6-dimethyl-3-ethoxycarbonyl-1,4-dihydropyridine-5-carbonyloxyacetate, sodium 2,6-dimethyl-3-methoxycarbonyl-1,4-dihydropyridine-5-carbonyloxyacetate and disodium 2,6-dimethyl-1,4-dihydropyridine-4-methoxycarbonyl-3,5-bis-(carbonyloxyacetate), were compared with already known medicaments, such as 1-methyl-2-phenylthiomethyl-3-carbethoxy-4-dimethylaminomethyl-5-oxybromoindole monohydrate hydrochloride and Oseltamivir, and results were represented in FIG. 1., FIG. 2., FIG. 3., FIG. 4. and FIG. 5.

It was surprisingly and unexpectedly that sodium 2,6-dimethyl-3-ethoxycarbonyl-1,4-dihydropyridine-5-carbonyloxyacetate, sodium 2,6-dimethyl-3-methoxycarbonyl-1,4-dihydropyridine-5-carbonyloxyacetate and disodium 2,6-dimethyl-1,4-dihydropyridine-4-methoxycarbonyl-3,5-bis-(carbonyloxyacetate) are effective against influenza by using it over a wide concentration range, see FIG. 3., FIG. 4., and FIG. 5.

1-12. (canceled)

13. A 2,6-Dimethyl-1,4-dihydropyridine-3,5-dicarboxylic acid ester type compound having general formula I 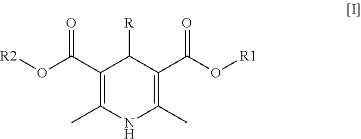
wherein
R is hydrogen or carboxylate-methyl ester;
R₁ is sodium carboxylate-methyl ester; and
R₂ is methyl, ethyl or sodium carboxylate-methyl ester.

14. The compound according to claim 13, which is sodium 2,6-dimethyl-3-ethoxycarbonyl-1,4-dihydropyridine-5-carbonyloxyacetate, having formula VI

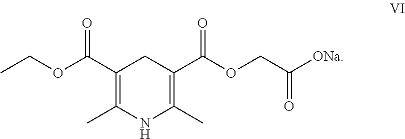

15. The compound according to claim 13, which is sodium 2,6-dimethyl-3-methoxycarbonyl-1,4-dihydropyridine-5-carbonyloxyacetate, having formula X

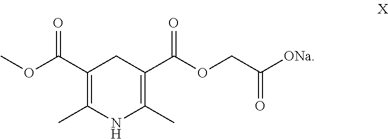

16. The compound according to claim 13, which is disodium 2,6-dimethyl-1,4-dihydropyridine-4-methoxycarbonyl-3,5-bis-(carbonyloxyacetate), having formula XV

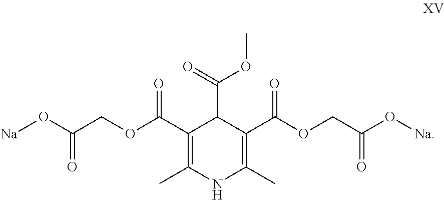

17. A process for preparing a compound according to claim 13, comprising the steps of:
a) reaction of a derivative of formaldehyde with a derivative of acetoacetic acid ester and enamine in an appropriate solvent;
b) treatment with sodium hydroxide in an appropriate solvent.

18. The process according to claim 17, wherein, in step a), the derivative of formaldehyde is formaldehyde or glyoxylic acid.

19. The process according to claim 17, wherein, in step a), the derivative of acetoacetic acid ester is 2-methoxycarbonyl methyl ester of acetoacetate or 2-ethoxycarbonyl methyl ester of acetoacetate.

20. The process according to claim 17, wherein, in step a), the derivative of enamine is methyl-β-aminocrotonate, ethyl-β-aminocrotonate or 2-ethoxycarbonyl methyl ester of β-aminocrotonate.

21. The process according to claim 17, wherein, in step a) and b), the appropriate solvent is selected from methanol, ethanol, propanol and butanol.

22. A pharmaceutical composition comprising as active ingredient a compound according to claim 13, optionally together with one or more pharmaceutically acceptable excipients.

23. A method of treating a condition requiring an antiviral agent in a subject in need thereof, comprising administering an effective amount of a compound according to claim 13.

24. A method of treating influenza in a subject in need thereof, comprising administering an effective amount of a compound according to claim 13.
